(19) United States
(12) Patent Application Publication
(10) Pub. No.: US 2024/0087715 A1
(43) Pub. Date: Mar. 14, 2024

(54) SURGICAL INSTRUMENT OPERATION MONITORING USING ARTIFICIAL INTELLIGENCE

(71) Applicant: McGinley Engineered Solutions, LLC, Casper, WY (US)

(72) Inventor: Joseph C. MCGINLEY, Casper, WY (US)

(21) Appl. No.: 18/261,823

(22) PCT Filed: Jan. 26, 2022
(86) PCT No.: PCT/US2022/013873

§ 371 Date: Jul. 17, 2023

Related U.S. Application Data
(60) Provisional application No. 63/142,856, filed on Jan. 28, 2021.

(51) Int. Cl. G16H 20/40; G16H 40/60; G16H 50/20

(52) U.S. Cl. CPC G16H 20/40; G16H 40/60; G16H 50/20

(57) ABSTRACT

Intelligent surgical instruments in which data is provided to a machine learning apparatus for use in monitoring the operation of the instrument. The machine learning apparatus may receive data from the instrument to be monitored such as from a measurement system thereof or one or more navigation sensors monitoring the instrument. In addition, data sources including demographic data, surgeon data, and patient data may also be provided by the machine learning apparatus. Such data may be provided by a secure protocol. In turn, the machine learning apparatus may analyze, in real-time, the data provided to the machine learning apparatus to monitor the surgical instrument. As such, the machine learning apparatus may assist in determining any appropriate output or condition regarding the surgical instrument based on the data analyzed.

### RELATED APPLICATIONS

This application claims the benefit of U.S. Provisional Application No. 63/142,856 filed on Jan. 28, 2021, entitled “SURGICAL INSTRUMENT OPERATION MONITORING USING ARTIFICIAL INTELLIGENCE”, the entirety of which is incorporated herein by reference.

### BACKGROUND

Powered surgical instruments are used pervasively in all surgical contexts, and especially in orthopedic surgery. Such powered surgical instruments may include drills, saws, burrs, pin drivers, or other powered instrument, which are typically powered electrically or pneumatically. In this regard, a number of operations including boring, sawing, grinding, or the like may be facilitated by surgical instruments.

Intelligent surgical instruments have been proposed that include onboard or remote sensors for monitoring instrument parameters to assist a surgeon in performing of an operation using an instrument. For instance, an instrument may include a measurement system that comprises force sensors, displacement sensors, optical sensors, or the like such as those described in U.S. Pat. Nos. 6,665,948, 9,370,372, 9,833,244, 10,758,250, 10,390,869, 10,321,921, 10,321,920, and U.S. patent application Ser. No. 16/305,353 (published as U.S. Pat. Pub. No. 2020/0113584), each of which is incorporated by reference herein in its entirety. Furthermore, external sensors remote from an instrument may be used to provide assistance or assistance data to a surgeon including those described in U.S. Pat. No. 10,806,525, which is incorporated by reference in its entirety.

In any regard, the processing of sensor input data to determine instrument placement, monitor instrument trajectory, monitor instrument performance, or provide other assistance data provides a valuable resource to a surgeon that may facilitate more efficient surgical operations and generally improve patient outcomes. However, as the number of sensors and sensor input complexity increases, the ability to effectively process sensor data to achieve meaningful outputs becomes more complex. Moreover, as each surgeon that utilizes an instrument may have a different technique in utilizing an instrument, providing a consistent output indicative of the desired monitored instrument parameter may be difficult.

As such, while intelligent surgical instruments may provide improved patient outputs, the need continues for more sophisticated and robust data processing approaches to provide enhanced functionality.

### SUMMARY

In turn, the present disclosure generally relates to use of artificial intelligence or machine learning in connection with intelligent surgical instruments. Specifically, the present disclosure contemplates systems in which various sensor inputs may be provided to a machine learning apparatus for real-time processing of the sensor inputs to derive meaningful outputs regarding the operation of the instrument. For example, the resulting outputs may relate to instrument placement, instrument operation, information regarding the patient upon which the operation is performed, or other meaningful data or outputs provided by the sensor data.

For example, the present disclosure may leverage different data sources to provide instrument analytics in real-time based on the specific operation being performed. Such data sources may include patient-specific data, surgeon-specific data, demographic derived data, or other specific data sources. For instance, a global neural network may be established across a plurality of hospitals or facilities that may capture and process global data based on demographic information. For example, global statistics aggregated across the network of hospitals or facilities may be used as an input to a machine learning apparatus to provide anticipated data regarding a specific patient on which an operation is performed such as the patient's bone density, physiological measurements, or the like. In an example, the exchange of data across multiple facilities may be provided according to a secure communication protocol including, for example, use of a blockchain protocol or the like.

The present disclosure may also utilize surgeon-specific data that may be captured regarding the surgeon utilizing the instrument. This may provide data to the machine learning apparatus specific to a surgeon based on their drilling technique. Such data may comprise historically derived sensor data specific to the surgeon and/or specific operation to be performed.

Further still, the machine learning apparatus may be integrated with surgical navigation devices. In this regard, the machine learning apparatus may process a plurality of input data including real-time sensor data and/or data from one or more of the network data sources to assist in predicting bone features as well as improve recognition of anatomy through navigational sensors such as visual sensors or other non-contact sensors.

The techniques described herein may be used in connection with a wide variety of instruments having different sensors integrated or paired with the instrument. For example, the instrument monitored may be provided according to any of the disclosures incorporated by reference above including, without limitation, instruments having force and displacement sensor, only a displacement sensor, only an accelerometer, having an onboard measurement system operated in conjunction with a surgical navigation system with remote sensors, or any other appropriate combination of instrument and sensor pairings. When used with a surgical navigation system, the machine learning apparatus may utilize data from navigation sensors (e.g., visual sensors, non-contact sensors, proximity sensors, time of flight sensors, etc.) to improve anatomy recognition and on the expectation of internal bone features.

The machine learning apparatus may employ artificial intelligence models that learn from the input data in real-time to fine tune one or more output triggers for the instrument. Such output triggers may include measured operation parameters (e.g., bore length or the like), instrument placement determination (e.g., including bicortical, unicortical, endosteal, subchondral instrument placement), patient anatomy measurement (e.g., bone density, physiological measurements, etc.), or other relevant outputs related to the surgeon, patient, instrument, and/or operation.

As described above, the present disclosure may leverage a plurality of input types, some of which may be shared among hospitals and/or other facilities in which an intelligent instrument is operated. As a diverse set of inputs may be provided across multiple facilities, the present disclosure also provides a secure network for exchange of input data for use in a machine learning apparatus to monitor instrument operation. For example, the secure network may utilize a blockchain or other cryptography structure to securely provide data across all systems. In this regard, the machine learning apparatus may receive data from a plurality of sources, yet still maintain security such that the platform may be non-corruptible, even with infinite base nodes. Furthermore, the cryptographic structure (e.g., blockchain) may provide further utility by facilitating smart contracts, inventory management, or other functions.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Other implementations are also described and recited herein.

### BRIEF DESCRIPTION OF THE DRAWINGS

FIG. 1 illustrates an example smart surgical system.

### DETAILED DESCRIPTION

While the invention is susceptible to various modifications and alternative forms, specific embodiments thereof have been shown by way of example in the drawings and are herein described in detail. It should be understood, however, that it is not intended to limit the invention to the particular form disclosed, but rather, the invention is to cover all modifications, equivalents, and alternatives falling within the scope of the invention as defined by the claims.

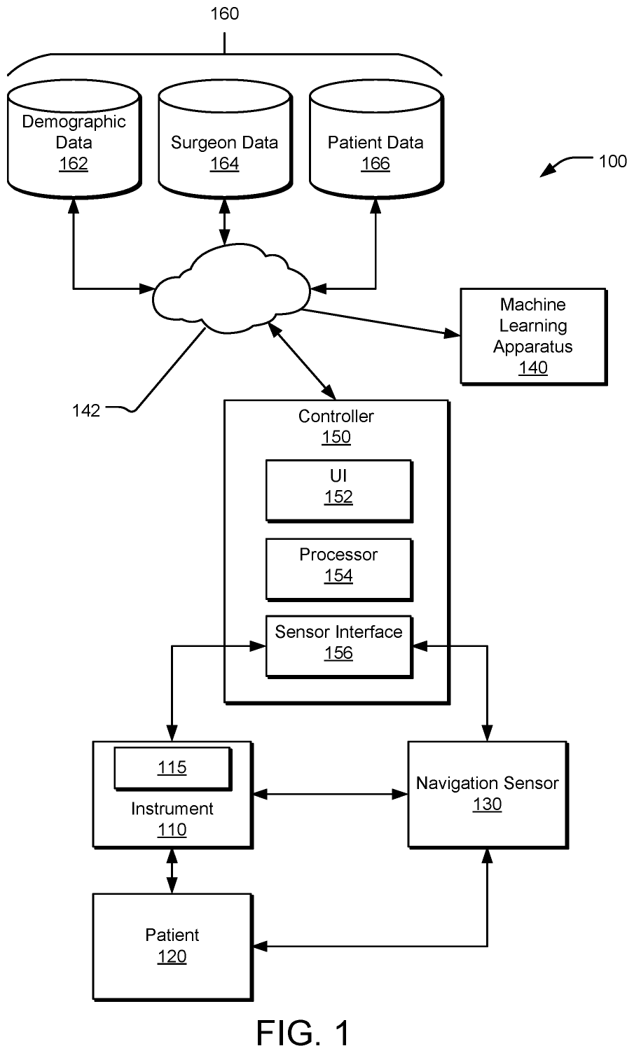

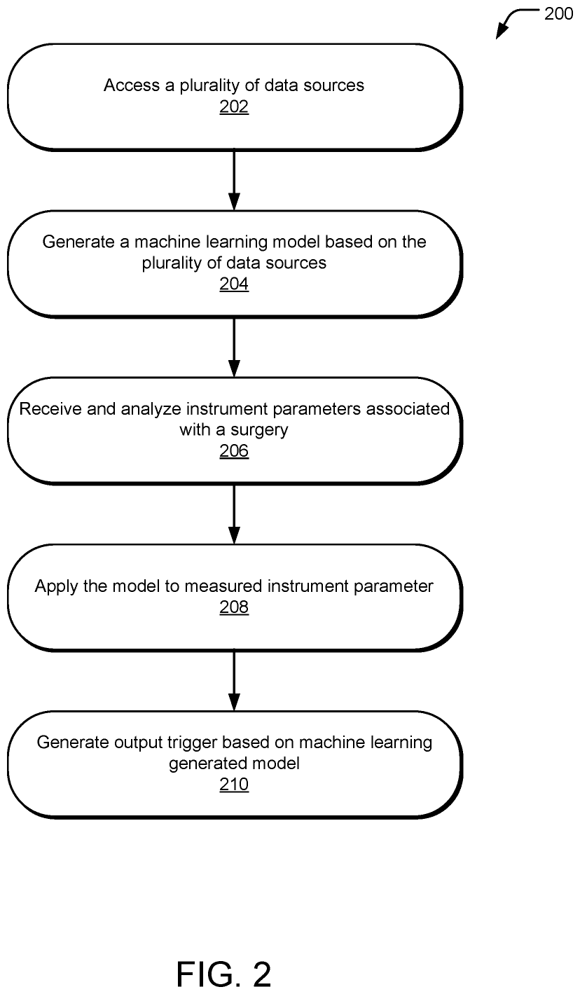

FIG. 1 illustrates an example intelligent surgical instrument system 100 of the present disclosure. The system 100 includes a powered surgical instrument 110. As described above, the powered surgical instrument 110 may be any appropriate type of instrument including, by way of example and not limitation, a drill, a pin driver, a saw, a burr, or a reamer. The instrument 110 may be manipulated by a human surgeon and/or may include robotic assistance. In the latter example, the instrument 110 may be partially automated by a robotic apparatus and/or may be fully controlled by a robotic surgical system. The instrument 110 may include a working portion or tool that acts upon a patient 120 to perform an operation such as a drilling operation, a cutting operation, a grinding operation, pin placement, or the like.

The instrument 110 may comprise a measurement system 115. In this regard, the measurement system 115 may monitor one or more instrument parameters 115 regarding the operation of the instrument 110 and/or conditions related to the working portion or tool. For instance, the measurement system 115 may comprise one or more onboard sensors capable of monitoring instrument and/or working tool parameters. Examples of such sensors include displacement sensors, force sensors, optical sensors, accelerometers, or the like. For example any of the measurement systems described in the material incorporated by reference above may be provided without limitation.

The instrument 110 may be in operative communication with a controller 150. For instance, the controller 150 may include a sensor interface 156 that is in operative communication with the instrument 110 to receive sensor inputs from the measurement system 115 regarding the operation of the instrument 110. The instrument 100 may be directly connected to the controller 150 by an appropriate hardwired interface including communications and/or power cabling. In another example, the instrument 110 may be in operative communication with the controller 150 via an appropriate wireless interface such as Bluetooth, Wi-Fi, Zigbee, or other wireless communication protocol.

In addition, the system 100 may include assisted surgical navigation such as described in the matters incorporated by reference above. As such, the system 100 may include one or more navigational sensors 130. The navigational sensors 130 are shown in FIG. 1 as being remote from the instrument 110. In this regard, the navigation sensors 130 may include cameras, time of flight sensors, LIDAR, infrared, or other appropriate sensor remote to the instrument 110 to monitor a position, orientation, trajectory, or other navigational data for the instrument 110. Also, while shown remote from the instrument 110, it may be appreciated that the measurement system 115 may also include navigational sensors such as cameras, time of flight sensors, LIDAR, infrared, or other appropriate sensor that are provided onboard the instrument 110 to monitor navigational parameters of the instrument 110. In any regard, the navigational sensors 130 may provide data to the controller 150 (e.g. by way of the sensor interface 156).

The controller 150 may also include a processor 154. The processor 154 may comprise one or more microprocessors and memory. The processor 154 may be in communication with or receive data from the sensor input 156. In this regard, and as described in various of the disclosures incorporated by reference above, the processor 154 may apply logic to the senor data received from the measurement system 115 and/or navigational sensors 130 to provide certain outputs or triggers related to the monitoring of the instrument 110. For example, the sensor data received by the controller 150 may provide information regarding the placement of the working tool of the instrument 110 relative to the anatomy of the patient 120. For example, a force and/or displacement sensor of the measurement system 115 may provide information regarding the placement of the leading edge of the working portion as the working portion is passed through various anatomy of the patient 120. In other examples, the measurement system 115 and/or navigation sensors 130 may monitor the instrument 115 to detect disengagement of the working tool from the patient 120 and/or unintentional accelerations of the working tool (e.g., such as when a drill bit passes through a bone with an increase in acceleration). Such disengagement or other unintended accelerations of the working tool may represent a danger to the patent 120. In other examples, the navigation sensors 130 may monitor the placement and/or trajectory of the instrument 120 to determine if the instrument 110 is in a correct position for performing a desired operation, potentially in reference to medical imaging correlated to the position of the patient 120. In each of the foregoing, feedback may be provided to a surgeon using the instrument 110 via a user interface 152 of the controller 150.

In each of the foregoing examples of monitoring operations that may be performed by the processor 154 of the controller 150, the processor 154 may monitor the sensor data received by the sensor interface 156 and apply logic to determine a signature within the sensor data that identifies a monitored condition or output as has been described in the disclosures incorporated by reference above. In prior approaches, such logic to identify a signature from the sensor data may rely on static, programmatic logic embedded in the controller to identify certain events or conditions from the sensor data for purposes of determining whether an event monitored for has occurred (e.g., such as placement of the working tool, “plunge” of a drill bit, disengagement of a working portion, etc.). While such predetermined, programmatic logic provides a genericized approach that is intended to be applicable to all patients, surgeons, and use conditions of the instrument, it has been recognized that variability within each of the patient anatomy, the technique of a surgeon, and/or a specific instrument used may provide variability regarding the occurrence of a given signature such that monitoring of the sensor data using a generic logic applicable to all contexts may not provide optimum performance over all scenarios. In turn, the present disclosure recognizes the inherent variability present in relation to performing and monitoring a surgical operation. In view of this variability, it has been recognized that the rote, predetermined logical definitions used to identify a signature of a monitored event in the sensor data may be preferentially supplemented or replaced by a machine learning apparatus that may adapt to variables present for a given operation to the monitored to more robustly and effectively monitor for a given condition during the operation of an instrument 110.

Furthermore, some surgical navigation systems may be used to identify anatomy of a patient. For example, surgical navigation systems may be used to assist in determining an instrument position or trajectory and/or to correlate anatomy relative to an instrument to surgical imaging data. Such approaches may use software approaches in an attempt to recognize anatomical structures and/or to correlate medical imaging data to observed anatomical features. Such functionality may also be benefited by application of a machine learning apparatus to assist in providing the functionality of the surgical navigation system by, for example, assisting in and/or fully developing recognition models. Further still, a machine learning apparatus may assist in and/or fully develop a correlative model for correlating medical imaging data to sensed anatomical features.

In view of the foregoing, the system 100 also includes a machine learning apparatus 140. As illustrated in FIG. 1, the machine learning apparatus 140 may communicate via network 142. The machine learning apparatus 140 may communicate with the controller 150 to, for example, receive sensor data from the controller 150. While shown in FIG. 1 as being in communication with the controller 150 via the network 142, it may also be appreciated that the machine learning apparatus 140 may be in direct communication with the controller 150 or integrated with the controller 150 (e.g., at the processor 154). In any regard, the machine learning apparatus 140 may also receive information from a number of other data sources 160. As will be discussed in greater detail below, the information received by the machine learning apparatus 140 from the controller 150 and/or the data sources 160 may be provided via a secure protocol.

The data sources 160 may comprise demographic data 162, surgeon data 164, and patient data 166, among other potential data sources. Each data source is described in greater detail below.

The demographic data 162 may include compiled data regarding a population of patients. The demographic data 162 may include historical operation data received in connection with other, prior operations performed on patients and/or measured data from a population of patients. In any regard, the demographic data 162 may include a statistical representation of certain parameters observed in relation to demographic data for the population of patients represented in the demographic data 162. As an example, in connection with a drilling operation performed on a patient 120 using the instrument 110, it may be known that bone density of the patient may be impactful on the resulting sensor data when monitoring an operation. Bone density information regarding the population of patients may be represented in the demographic data 162 such that the demographic data 162 may present a statistical representation of bone density relative to demographics for the population of patients. As will be described in greater detail below, by cross referencing a given patient's demographics to the demographic data 162, a given patient's bone density may be estimated or predicted in view of the demographic data 162. While bone density is provided as an example, other meaningful characteristics useful in monitoring an operation may be provided in the demographic data 162 without limitation.

The data sources 160 may also include surgeon data 164 that may include historical data regarding a given surgeon's performance in performing and operation. For example, historical force profiles and or displacement profiles regarding the manner in which a given surgeon performs an operation may be recorded in the surgeon data 164. As such, intricacies or particularities of a given surgeon may be captured in the surgeon data 164. The surgeon data 164 may be provided to the machine learning apparatus 140 to assist in tailoring monitoring of the monitored condition based on the historic data regarding the specific surgeon operating the instrument 110.

Further still, the data sources 160 may include patient data 166. The patient data 166 may be data regarding the patient 120 on which an operation is performed by the instrument 110. As described above, others of the data sources 160 may relate to information regarding the patient 120. Thus, the patient data 166 may be accessed regarding the patient 120 to allow cross referencing of relevant data to the patient 120 undergoing the operation.

As such, the machine learning apparatus 140 may have data source at least including the controller 150 for providing sensor data from the measurement system 115 and/or navigation sensors 130 and the data sources 160 that may include demographic data 162, surgeon data 164, and/or patient data 166. From these data sources, the machine learning apparatus 140 may be employed to determine the occurrence of a monitored condition in real-time during the operation of the instrument 110. As described above, the monitored condition may be any appropriate trigger or output discussed in any of the references incorporated by reference herein. However, rather than use of rote, preprogrammed logic, the machine learning apparatus 140 may analyze the data provided from the various data sources 160 and/or controller 150 to determine particular instance of a measured event as determined by the machine learning apparatus 140 in view of the data provided. Further still, the machine learning apparatus 140 may be used to more accurately determine or recognize patient anatomy by a surgical navigation system to help determine a position, trajectory, or location of the instrument 110 by the surgical navigation system.

As noted above, the data sources 160 and/or data from the controller 150 may be provided to the controller 150 in a secure manner. One example of a secure protocol for exchange of such data may include a blockchain technology. Data may be provided to the machine learning apparatus 140 as blocks securely included in a blockchain. Moreover, as each operation is conducted using an intelligent instrument 110, the data utilized or processed may be appended to the blockchain for further use by a machine learning apparatus 140 in later operations. Thus, each operation conducted using a machine learning apparatus 140 may generate further data to be used by machine learning apparatuses 140 in later operations. Thus, while sensitive data (e.g., PHI, HIPAA data, etc.) may be redacted or removed from the data, the data may be made available to systems 100 for use in later operations. Moreover, such information may be shared across a plurality of facilities. Thus, the system 100 may become more robust, efficient, and accurate with each use across the plurality of facilities.

The machine learning apparatus 140 may comprise any appropriate machine learning or artificial intelligence technology. For example, the machine learning apparatus 140 may employ an artificial neural network comprising inputs as discussed above with identified outputs to be recognized from the data provided. A number of hidden layers having a given number of hidden nodes may be provided in the neural network. Other supervised or unsupervised approaches to machine learning may be applied without limitation. In a supervised context, historical log data from operations including, potentially, post-operative confirmation data may be used as training data against which the machine learning module 140 may be trained.

With further reference to FIG. 2, example operations 200 are depicted of a process for use of a machine learning model in connection with operation of a powered surgical instrument to provide actionable trigger outputs or other actionable data to a user of the surgical instrument. The operations 200 include an accessing operation 202 in which a machine learning apparatus accesses data from a plurality of data sources. As described above, such data sources may include demographic data, surgeon data, and/or patient data that may be provided via a secure manner in compliance with regulatory and other privacy concerns. The data sources may additionally or alternatively include other types of information without limitation including, for example, historical surgical outcomes, clinical information, genetic information, or any other potential source of data that may provide correlative or causative indications with respect to a surgical operation.

A generating operation 204 includes generating a machine learning model based on the plurality of data sources accessed in the accessing step 202. The generating operation 204 may utilize any appropriate machine learning approach including supervised or unsupervised learning models to provide actionable output data or other parameters. In turn, the operations 200 may include a receiving operation 206 in which instrument parameters associated with a surgery may be received in real time. The received instrument parameters for a surgery occurring in real time may be received for real-time determination of one or more of the output triggers as discussed below.

The operations 200 may include an applying operation 208 in which the machine learning generated model may be applied to the received instrument parameters. Thereafter, generating operation 210 may include generating one or more output trigger base on the machine learning generated model in view of the received instrument parameters. As discussed above, the output trigger may including one or more different actionable items of data that include, for example, information regarding instrument placement; status of the instrument with respect to anatomy; navigational information regarding the position, orientation, and/or trajectory of the instrument with respect to patient; or any other actionable data than provided real-time to a surgeon to allow for feedback regarding the surgical operation occurring respect to the patient.

While the invention has been illustrated and described in detail in the drawings and foregoing description, such illustration and description is to be considered as exemplary and not restrictive in character. For example, certain embodiments described hereinabove may be combinable with other described embodiments and/or arranged in other ways (e.g., process elements may be performed in other sequences). Accordingly, it should be understood that only the preferred embodiment and variants thereof have been shown and described and that all changes and modifications that come within the spirit of the invention are desired to be protected.

What is claimed is:

1. A smart surgical system comprising:
a surgical instrument comprising a working tool;
a measurement system of the surgical instrument for determining at least one working parameter of the surgical instrument;
a plurality of data sources external from the surgical instrument; and
a machine learning apparatus operative to receive data from the measurement system and the plurality of data sources to identify an output regarding the operation of the surgical instrument.

2. The system of claim 1, wherein the plurality of data sources comprise at least demographic data, surgeon data, and patient data.

3. The system of claim 2, wherein the plurality of data sources comprise historical data.

4. The system of claim 1, wherein the measurement system comprises at least a force sensor and a displacement sensor.

5. The system of claim 1, wherein the output comprises at least one of a placement of the working tool portion of the instrument, a location of the instrument, or a trajectory of the instrument.

6. The system of claim 1, wherein at least the plurality of data sources are provided to the machine learning apparatus via a secure protocol.

7. The system of claim 6, wherein the secure protocol comprises a blockchain protocol.

8. A method for use of machine learning in connection with use of a powered surgical, comprising:
accessing a plurality of data sources;
generating a machine learning model based on the plurality of data sources that characterizes an operation performed by a surgical instrument;
receiving at least one working parameter measured from the surgical instrument; and
applying the machine learning model to the at least one working parameter to determine an output regarding the operation performed by the surgical instrument.

9. The method of claim 8, wherein the plurality of data sources comprise at least demographic data, surgeon data, and patient data.

10. The method of claim 9, wherein the plurality of data sources comprise historical data.

11. The method of claim 8, further comprises:
measuring the at least one working parameter using at least a force sensor and a displacement sensor.

12. The method of claim 8, wherein the output comprises at least one of a placement of the working tool portion of the instrument, a location of the instrument, or a trajectory of the instrument.

13. The method of claim 8, wherein the accessing the plurality of data sources include using a secure protocol.

14. The method of claim 13, wherein the secure protocol comprises a blockchain protocol.
